(19) United States
(12) Patent Application Publication
(10) Pub. No.: US 2012/0121676 A1
(43) Pub. Date: May 17, 2012

(54) METHOD FOR MANUFACTURING CAPSULES, RESULTING CAPSULES, AND USE OF SAID CAPSULES

(75) Inventors: Gladys Massiera, Montpellier (FR); Etienne Loiseau, Millau (FR); Manouk Abkarian, Montpellier (FR)

(73) Assignees: UNIVERSITE MONTPELLIER 2- SCIENCE ET TECHNIQUE; Centre National de la Recherche Scientifique, Paris (FR)

(21) Appl. No.: 13/384,756

(22) PCT Filed: Jul. 20, 2010
(86) PCT No.: PCT/FR10/00523

§ 371 Date: Jan. 18, 2012

(30) Foreign Application Priority Data
Jul. 20, 2009 (FR) FR 09 55035

(51) Int. Cl. A61K 9/50; A61K 38/17; B01J 13/04; A61K 38/16; A61K 31/7088; A61P 7/00; A61K 9/127; A61K 8/11

(52) U.S. Cl. 424/401; 424/451; 424/450; 514/13.5; 514/1.1; 514/44 R; 264/4.1; 425/5

(57) ABSTRACT

The invention relates to a one-step process for manufacturing capsules from 1 to 100 microns with a polydispersity index of less than 10%, to suspensions of capsules obtained via this process, and to the use of these suspensions of capsules.

The present invention relates to the field of manufacturing capsules that are useful especially for encapsulating a sample of interest or as a tool for calibrating analysis material.

Several methods for producing capsules have already been described in the literature. For example, for a type of capsules whose envelope is a bilayer, hydration methods exist, in particular electroformation, which consist in hydrating a lipid film and which allow the production of capsules of good quality, but in a very poor yield and with a large polydispersity of the size of the capsules obtained. Methods also exist for the production of a double emulsion followed by evaporation of the solvent included between the two amphiphilic monolayers; or alternatively methods for producing a microfluidic-calibrated emulsion and then dispersing the emulsion produced in an aqueous solution of water and ethanol.

However, none of the preceding methods makes it possible to manufacture, within short time periods, large amounts of capsules of controlled size and of good quality, i.e. defect-free spheres, with good reproducibility of the method. Moreover, a large number of the prior-art methods involve organic solvents, which are often toxic, and which need to be removed before the capsules can be used.

The Applicant became particularly interested in a method described in Pautot et al., Langmuir 2003, 19, 2870-2879, in which a reverse emulsion is prepared by mixing an aqueous phase and a first oily phase in the presence of surfactant: the surfactant molecules present in the oil become adsorbed onto the surface of the aqueous drop and form a monolayer. A double phase formed from an aqueous phase, an interface and an intermediate phase is then prepared as follows: a second oily phase also supplemented with surfactant is poured onto an aqueous phase, and an interface is allowed to form. Since the oil is less dense, it remains above the water. The emulsion initially prepared is poured onto this double phase, and the vesicles, which are heavier than the oily phase, sediment towards the aqueous phase. On crossing the interface, they become covered with a second lipid layer. In PNAS 2003, 19, vol. 100, 10718-10721, Pautot et al. describes the manufacture of vesicles with an asymmetric membrane from the same type of emulsion as that mentioned above, and from the same type of intermediate phase, followed by the application of a centrifugal force at 120×g for 10 minutes to transfer the water droplets across the oil/water interface to the lower aqueous phase.

However, the method of Pautot et al., with or without centrifugation, does not make it possible to obtain suspensions of vesicles with satisfactory polydispersity. Moreover, the methods of Pautot et al. are not sufficiently reproducible to be able to be used industrially.

In the field of manufacturing capsules, it is known that the productivity of the method is an important industrial factor. Industrially efficient and reproducible processes, which make it possible to control the core of the capsule and the formation of the capsules themselves, in their nature, their quality, their quantity and their polydispersity index so as to control the production, are thus still sought.

One subject of the invention is thus a novel process for manufacturing capsules, which is a robust, economical one-step process that allows fine control of the size of the capsules produced and a high yield. The process according to the invention makes it possible to obtain capsules that are free of defects. The use of the process according to the invention avoids any coalescence of the capsules.

More specifically, the invention relates to a one-step process for manufacturing capsules from 1 to 100 microns, with a polydispersity index of less than 10%, comprising the placing in contact of droplets of homogeneous size of an aqueous composition, emitted continuously, with an intermediate phase that is in a rotating chamber, said chamber comprising an aqueous phase and an intermediate phase, these two phases forming an interface, through which said droplets are forced under the effect of the centrifugal force generated by the rotation of the chamber, followed by the recovery of an aqueous suspension of capsules. Under the effect of the centrifugal force, the interface is vertical. Advantageously, the intermediate phase is a dispersion of amphiphilic molecules in a water-immiscible solvent with a density less than that of water.

According to one preferred embodiment of the invention, the aqueous phase has a density of at least 0.5 to 25 g/l lower than the density of the aqueous composition; advantageously, the aqueous phase and the aqueous composition are isoosmotic. In particular, the aqueous composition is injected via capillaries at a flow rate of 100 to 500 μl/h and preferably 150 to 250 μl/h or at a fixed pressure of 80 to 500 mbar and preferably 100 to 200 mbar (depending on the size of the capillary) into the intermediate phase that is in the rotating chamber, at a fixed and determined distance from the interface, said injection resulting, via the rotation, in the production of droplets at regular intervals in the intermediate phase via a dropwise mechanism or a jet mechanism depending on the size of the drops that it is desired to produce. The polydispersity of the droplets produced is, according to the process of the invention, dependent on the size of the capillary and on the capillary number of the flow at the end of the capillary, this number being less than 1, preferably from 10⁻⁴ to 10⁻¹ and most preferentially about 0.5×10⁻³.

In another embodiment of the invention, the aqueous phase will be injected from a perforated cylinder making it possible to multiply the number of injection points and thus the yield.

A subject of the invention is also a device for performing the process according to the invention, this device comprising: a chamber in which the capsules will be formed; this chamber being able to be rotated, said chamber comprising an aqueous phase and an intermediate phase, and means for placing the aqueous composition in contact with the contents of the chamber.

A subject of the invention is also a suspension of capsules that may be obtained via the process of the invention. In one particular embodiment of the invention, the capsules may be sedimented onto a substrate to form a network resembling an artificial fabric. Preferably, this artificial fabric is a lawn of vesicles stuck together. These capsules may contain actin filaments. This fabric is obtained from the process according to the invention, by sedimentation of the suspension directly obtained via the process according to the invention.

Advantageously, the capsules of the capsule suspension according to the invention comprise an envelope and a core of aqueous composition. In a first embodiment, the thickness of the envelope is between 1 nm and 10 microns, preferentially between twice the size of the dispersed amphiphilic molecule and 10 microns.

In a first embodiment in which the envelope is a lipid bilayer, the thickness of the envelope is between 1 and 100 nm and most preferentially from 5 to 20 nm; in a second embodiment in which the envelope is formed from an intermediate phase, the thickness of the envelope is less than or equal to the radius of the core, and is preferably between 100 nm and 10 microns. In this second embodiment, the volume of the envelope may have 3 to 10 times and preferably about 7 times the volume of the core.

A subject of the invention is also the use of the process according to the invention for encapsulating aqueous compositions comprising or formed by pharmaceutical active principles, cosmetic active agents, biological substances, for example nucleic acids, proteins, colloidal solutions, human or environmental biological samples, or alternatively for encapsulating blood products. A subject of the invention is a capsule suspension that may be obtained via the process of the invention, said capsules encapsulating pharmaceutical active principles, cosmetic active agents, nucleic acids, proteins, human or environmental biological samples, or alternatively blood products (suspension of hemoglobin or of blood substitute, or any labile or stable blood product, especially of red blood cell concentrate, platelet concentrate or plasma type; or alternatively a blood-derived medicament especially of the type such as coagulant proteins, immunoglobulins, albumin).

In particular, a subject of the invention is the use of the process according to the invention for the production of artificial blood products, especially blood substitutes. In a first embodiment, the envelope is preferably a gas-permeable and hemoglobin-impermeable bilayer. A subject of the invention is a capsule suspension that may be obtained via the process of the invention, said capsules encapsulating hemoglobin and the envelope of said capsule being gas-permeable and hemoglobin-impermeable.

A subject of the invention is also the use of a suspension of capsules according to the invention as a calibration tool. Specifically, the process according to the invention allows perfect control of the size and content of the capsules, thus making it possible to use capsule suspensions according to the invention as a calibration tool.

For the purposes of the present invention, the term: “capsule” refers to a sphere limited by an envelope that may contain an aqueous composition, this sphere having a diameter of 1 to 100 microns, preferably 5 to 80 microns and more preferentially from 10 to 30 microns; in one particular embodiment, the capsule according to the invention is a vesicle, i.e. a capsule whose envelope is an amphiphilic bilayer, preferably a lipid bilayer;

“polydispersity” refers to the size distribution of a population of capsules. This is obtained by image analysis, by detecting the contours of the capsules and by adjusting them with a circle. The histogram of the diameters of the circles makes it possible to obtain the mean and the distribution variance, the variance-to-mean ratio defining the polydispersity index. The smaller this ratio, the narrower the distribution and the more the capsule solution approaches a monodisperse capsule solution; “intermediate phase” refers to a liquid composition, which may be composed of several fluids of different masses per unit volume, said intermediate phase being: of mass(es) per unit volume less than that of the aqueous composition and than that of the aqueous phase, immiscible with the aqueous composition and/or the aqueous phase, comprises or is formed from amphiphilic molecules, which may be chosen especially from lipids, di-, tri- or multiblock polymers, surfactants and proteins.

Thus, a subject of the invention is a one-step process for manufacturing capsules or a suspension of capsules of 1 to 100 microns, with a polydispersity index of less than 10%, preferably less than 9%, preferentially less than 7% and most preferentially of about 6%. Advantageously, the process is performed continuously, the aqueous composition is injected from the capillary into the intermediate phase with a pressure of 80 mbar to 500 mbar, preferentially from 100 to 200 mbar, and the droplets of aqueous composition are pulled from the end of the capillary by the force associated with the rotation of the liquid (the intermediate phase) with which the end of the capillary is in contact, located in the chamber. Thus, the droplets arrive sequentially into the intermediate phase.

This intermediate phase is such that the flow regime of the fluid around the capillary is of low capillary number (less than 1), which ensures a dropwise regime of the aqueous composition, this regime being dominated by the interface tension between the aqueous composition and the intermediate phase, and thus great reproducibility of the drop size, i.e. a small polydispersity index of the drops of aqueous composition.

Each drop pulled from the capillary is subjected to the centrifugal force and rapidly becomes distanced from the capillary towards the interface: thus, coalescence phenomena are avoided, or at the very least minimized.

According to a first embodiment, the intermediate phase comprises or consists of two layers of fluids, with different densities: a first layer that is less dense and less viscous than the second, makes it possible to maintain a low capillary number so that the dropwise mechanism of the aqueous composition is dominated by the interface tension, thus making it possible to obtain an optimized size distribution; a second layer, which is a dispersion of amphiphilic molecules.

According to a second embodiment, an intermediate phase comprising a single layer, that of the dispersion of amphiphilic molecules, preferably of lipids, will be used.

The thickness of the intermediate phase and the concentration of amphiphilic molecules of this one-layer intermediate phase, or of the second layer of the bilayer intermediate phase, are two dependant parameters. Thus, the concentration of amphiphilic molecules sets the adsorption time of the amphiphilic molecules onto the drops of aqueous composition up to saturation. The thickness of this layer of intermediate phase will thus be chosen such that the drops formed from the capillary flying in the intermediate phase have the time to become covered to the point of saturation with amphiphilic molecules before reaching the interface. According to a preferred embodiment of the invention, the concentration of amphiphilic molecules is from 0.05 to 5 mM, preferably 0.1 to 1 mM and most preferentially about 0.5 mM.

In accordance with the process according to the invention, the aqueous composition may be any composition of interest, especially of the type containing biological substances, for example nucleic acids, proteins, human biological samples (red blood cells, white blood cells, platelets, etc.) or environmental samples, pharmaceutical active principles, cosmetic active agents, colloidal solutions, etc. According to one particular embodiment of the invention, the aqueous composition is a blood product, preferably a suspension of hemoglobin or of blood substitute, or any labile or stable blood product, especially of the red blood cell concentrate, platelet concentrate or plasma type; or alternatively a blood-derived medicament of the type especially such as coagulant proteins, immunoglobulins, albumin.

According to one embodiment of the invention, the aqueous composition comprises lipids; this embodiment is preferred when the lipid used is more soluble in the aqueous composition than in the intermediate phase.

In a first embodiment, the intermediate phase is a dispersion of amphiphilic molecules, preferably of lipids, in a composition whose density is less than that of water, which may especially be a mineral oil or a mixture of mineral oils, an alkane or a mixture of alkanes, an alkene or a mixture of alkenes, a terpene or a mixture of terpenes, or other solvents such as chloroform, toluene or an alcohol (methanol or ethanol). Advantageously, the composition is decane, hexadecane, dodecane or squalene. According to one preferred embodiment of the invention, the lipids are dried prior to being dispersed in the oil, so as to be as water-free as possible. Preferably, the amphiphilic molecules are dispersed in the oil by sonication.

According to a particular embodiment, the intermediate phase is a dispersion of lipids in an oil whose density is less than that of water, said lipids being placed in an oil-miscible solvent and the whole is then mixed with the oil, said dispersion then being evaporated to remove the solvent and any traces of water.

According to one particular embodiment of the invention, the aqueous phase is a saline solution or physiological saline, or a solution comprising at least one sugar. Advantageously, the aqueous phase is a glucose solution. According to a preferred embodiment, the aqueous phase is isoosmotic with the aqueous composition.

According to a preferred embodiment of the invention, the aqueous composition, which will become the core of the capsule, is placed in contact with the intermediate phase by injection of droplets of this composition, these drops being of controlled and homogeneous size, at a set and determined distance from the interface.

According to a particular embodiment of the invention, the means for injecting drops of aqueous composition of controlled size into the intermediate phase is one or more capillaries, of 2 to 50 microns. Advantageously, these capillaries are formed using a micropipette draw and the exterior of the capillary is then made hydrophobic by any appropriate means. According to one particular embodiment of the invention, the end of the capillary is introduced into the intermediate phase close to the air-intermediate phase interface and the aqueous composition contained in the capillary is injected at a rate of 100 to 500 μl/h, preferably 250 μl/h.

The injected droplets are subjected to the centrifugal force due to the rotation of the chamber, and, under the action of this centrifugal force, follow a path in the chamber, known as the “flight”. According to one embodiment of the invention, during the flight, the droplets become covered with amphiphilic molecules dispersed in the intermediate phase. The flight time, i.e. the time for the droplet to pass in the intermediate phase between the moment at which it is pulled from the capillary up to the moment at which it comes into contact with the interface, is controlled by the centrifugal force. The flight time also depends on the radius of the droplet, the viscosity of the intermediate phase, and the thickness of the intermediate phase. Controlling the flight time makes it possible to obtain good capsule quality, i.e. spherical capsules free of envelope defects.

Thus, according to the invention, the process is optimized as a function of the size of the chamber, and as a function of the flight time necessary for good coverage of the droplet. Thus, the determination of the optimized speed of rotation of the chamber is determined by successive tests, which are very simple for a person skilled in the art to perform.

The capsules end the flight when they come into contact with the interface, and then they cross the interface: during this crossing, in a first embodiment, they become covered with a second layer of amphiphilic molecules, to form amphiphilic bilayer capsules.

In a second embodiment, the capsule entrains intermediate phase during its crossing of the interface to form thick capsules.

Once the interface has been crossed, the capsules are in the aqueous phase, from which they are recovered, via any adequate means.

Advantageously, the process according to the invention is a high-yield process, i.e. it allows a production frequency of 1 to 1000 Hz and preferably from 500 to 1000 Hz. Higher frequencies may be obtained by simple adaptation of the process.

The droplet emission frequency is optimized as a function of the flight time and of the interface passage time, to avoid coalescence, especially in flight and at the surface of the interface.

In the embodiment of the invention using capillaries, the drops are pulled from the capillaries at regular intervals.

The centrifugal force has the effect of varying the thickness of the envelope; it also determines the flight time and the time of passage through the interface, and, finally, has an influence on the passage through the interface per se.

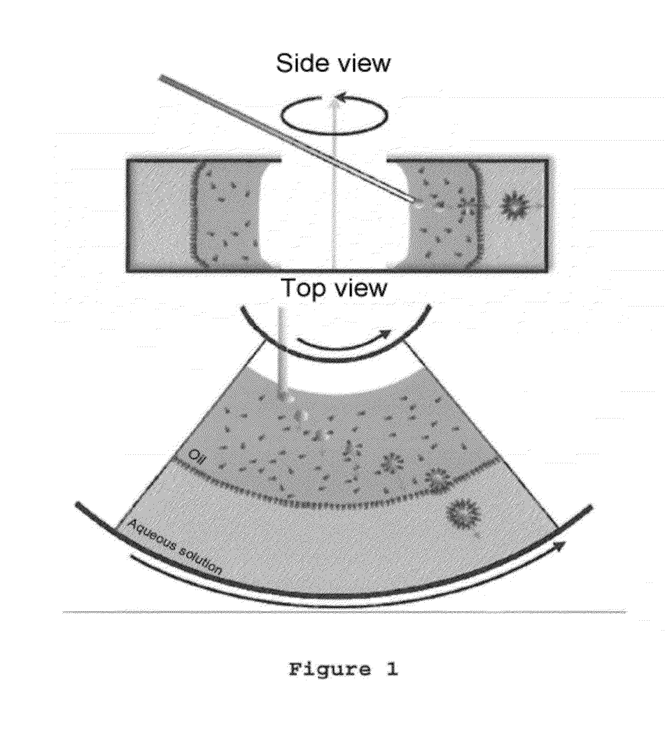

The invention may be understood more clearly on reading the description that follows, which illustrates the invention in a nonlimiting manner and is read with regard to FIGS. 1, 2 and 3.

FIG. 1 is a scheme of the chamber for manufacturing the capsules in top view and in side view and is read with reference to Example 1.

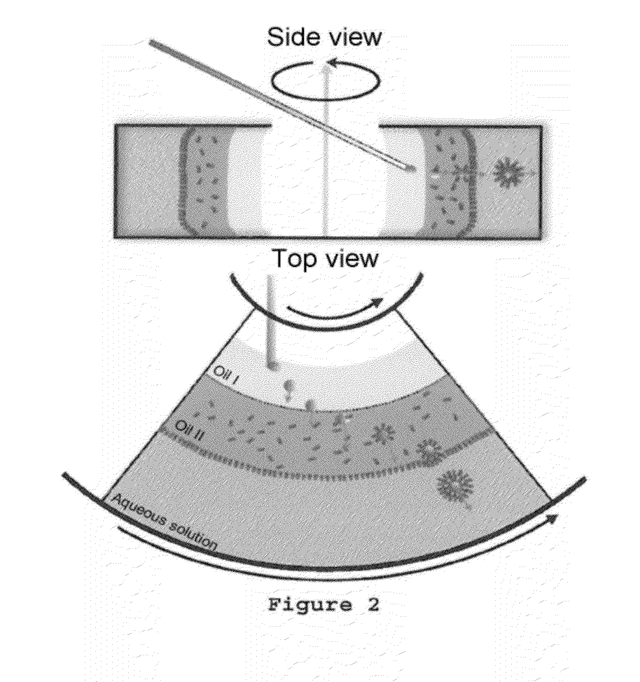

FIG. 2 is a scheme of the chamber for manufacturing the capsules in top view and in side view and is read with reference to Example 2.

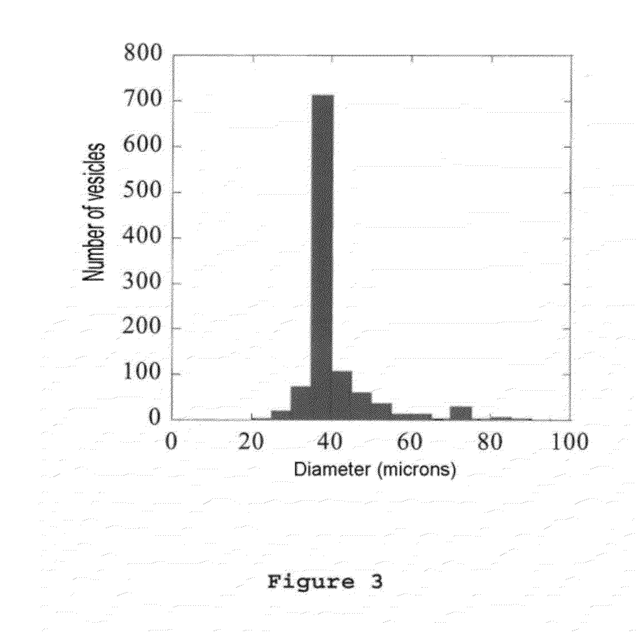

FIG. 3 is a graph showing the polydispersity of capsule suspensions that may be obtained via the process according to the invention.

### EXAMPLE 1

### Dissolution of the Lipids and Evaporation of the Traces of Water

The lipids (egg phosphatidyl choline) are dissolved in 2 ml of methanol and then evaporated under a pressure of 200 mbar for 5-10 minutes at a temperature of 40° C. Once the flask is lined with a homogeneous lipid film, the evaporation is continued for one hour at a pressure of 100 mbar (and a temperature of 40° C.). The mineral oil (Sigma® M3516) is then added to the lipids (egg phosphatidyl choline) at a concentration of 0.5 mM. To disperse the lipids, the solution is sonicated in a bath at a temperature of 40° C.

Preparation of the Capillaries

Capillaries between 2 and 50 microns are formed using a micropipette draw and are then silanized by dipping the capillary tip in a solution of silane (0.1% [3-(trimethoxysilyl)propyl]octadecyldimethylammonium chloride is added to 90% methanol, 10% water mixture) for 2 minutes using a stream of nitrogen via the capillary to prevent its interior from becoming silanized. This silanization has the function of making the exterior of the capillary hydrophobic.

Description of the Chamber

The chamber in which the capsules are formed is composed of a Petri dish 4 cm in diameter hermetically sealed by bonding, and whose upper part is equipped with a 1 cm orifice for introducing the capillary.

Production of the Capsules

The chamber is attached to a rotating motor (in this case a motor for spinning from 5 to 70 rps). The chamber is rotated at 10 rps, and is then successively filled with 1.5 ml of glucose solution, 5 ml of lipid solution, which instantaneously form a vertical interface on account of the centrifugal force. The capillary is then introduced into the solution of lipids in the oil, close to the air-oil interface and a sucrose solution is then injected using the capillary at a pressure ranging from 80 mbar to 500 mbar or in an equivalent manner, at a rate of 250 μl/h.

FIG. 1 shows a chamber containing the two layers of fluids (aqueous solution, oil). The chamber is rotated about its axis of revolution at a frequency that may range from 5 to 70 rps. The fluids are thus in the form of superposed vertical layers, from the most dense (the most eccentric) to the least dense. FIG. 1 also shows the principle for formation of the capsules according to the invention: drops are first produced by injection of an aqueous composition into the intermediate phase in rotational motion, and the drop is then forced by the centrifugal force across the intermediate phase-aqueous phase interface, to become a capsule.

### EXAMPLE 2

FIG. 2 shows one particular embodiment of the invention, which uses an additional layer of oil, which is less dense and less viscous than the other layer of oil, which provides a dropwise production dominated by the interface tension (capillary regime).

Thus, for a very narrow size distribution, an additional layer of 1.5 ml of another oil (decane) was used, with a viscosity and a density less than those of the mineral oil and into which was injected the sucrose solution. A lower viscosity ensures that the dropwise production of the droplets is governed by the interface tension. For example, the capillary number for water droplets released into the mineral oil at 30 rps is equal to 0.14, whereas it is only 0.004 if the drops are injected into decane. The droplets are formed sequentially, covered with the lipids during their travel (flight) through the layer of lipid solution, before crossing the oil solution-glucose interface, during which they become covered with an additional layer of lipids and then become capsules, in our case filled with sucrose, and dispersed in glucose. FIG. 3 indicates the size distribution of the capsules obtained with a 10 micron capillary, a speed of 30 rps (×40 g), and injection from the capillary in decane at 400 mbar.

1. A one-step process for manufacturing capsules from 1 to 100 microns, with a polydispersity index of less than 10%, comprising:
the placing in contact of droplets of homogeneous size of an aqueous composition, emitted continuously, with an intermediate phase that is in a rotating chamber, said chamber comprising an aqueous phase and an intermediate phase, these two phases forming an interface, through which said droplets are forced under the effect of the centrifugal force generated by the rotation of the chamber;
followed by the recovery of an aqueous suspension of capsules.

2. The process as claimed in claim 1, wherein the intermediate phase is a dispersion of amphiphilic molecules in a water-immiscible solvent with a density less than that of water.

3. The process as claimed in claim 1, wherein the aqueous phase has a density less than that of the aqueous composition and the aqueous phase and the aqueous composition are isoosmotic.

4. The process as claimed in claim 1, wherein the aqueous composition is injected via capillaries at a flow rate of 100 to 500 μl/h or at a fixed pressure of 80 to 500 mbar into the intermediate phase that is in the rotating chamber, at a fixed and determined distance from the interface, said injection resulting in the production of droplets at regular intervals in the intermediate phase.

5. The process as claimed in claim 1, wherein the polydispersity of the droplets produced is dependent on the capillary number of the flow at the end of the capillary.

6. A device for performing the process as claimed in claim 1, comprising:
a chamber in which the capsules will be formed; this chamber being able to be rotated, said chamber comprising an aqueous phase and an intermediate phase,
means for placing the aqueous composition in contact with the contents of the chamber.

7. A suspension of capsules that may be obtained via the process as claimed in claim 1.

8. The suspension of capsules as claimed in claim 7, wherein said capsules comprise an envelope and a core of aqueous composition, in which the thickness of the envelope is between 1 nm and 10 microns.

9. The suspension of capsules as claimed in claim 8, wherein said envelope is a lipid bilayer.

10. The suspension of capsules as claimed in claim 8, wherein said envelope is formed from an intermediate phase.

11. The suspension of capsules as claimed in claim 8, wherein said capsules encapsulate hemoglobin and the envelope of said capsules are gas-permeable and hemoglobin-impermeable.

12. The suspension of capsules as claimed in claim 8, wherein said capsules encapsulate pharmaceutical active principles, cosmetic active agents, nucleic acids, proteins, human or environmental biological samples, or alternatively blood products.

13. (canceled)
